(19) United States
(12) Patent Application Publication
(10) Pub. No.: US 2025/0162987 A1
(43) Pub. Date: May 22, 2025

(54) PREPARATION OF GAMMA-GLUTAMYLCYSTEINE, A PRECURSOR OF GLUTATHIONE

(71) Applicants: CHILDREN'S NATIONAL MEDICAL CENTER, Washington, DC (US); VIRGINIA COMMONWEALTH UNIVERSITY, Richmond, VA (US)

(72) Inventors: Frank GUPTON, Richmond, VA (US); Marshall L. SUMMAR, Washington, DC (US); Perrer TOSSO, Richmond, VA (US); Kashinath KOMIRISHETTY, Richmond, VA (US)

(73) Assignees: CHILDREN'S NATIONAL MEDICAL CENTER, Washington, DC (US); VIRGINIA COMMONWEALTH UNIVERSITY, Richmond, VA (US)

(21) Appl. No.: 18/840,524

(22) PCT Filed: Apr. 4, 2023
(86) PCT No.: PCT/US2023/017374

§ 371 Date: Aug. 22, 2024

Related U.S. Application Data
(60) Provisional application No. 63/326,959, filed on Apr. 4, 2022.

(51) Int. Cl. C07C 319/20; C07C 319/12

(52) U.S. Cl. CPC C07C 319/20; C07C 319/12

(57) ABSTRACT

A method to prepare γ-L-Glutamyl-L-Cysteine or bis-γ-L-Glutamyl-L-Cystine including direct reaction of cysteine or cystine with Di-tert-butyl (S)-(2,6-dioxotetrahydro-2H-pyran-3-yl)iminodicarbonate (formula 5) is provided (I).

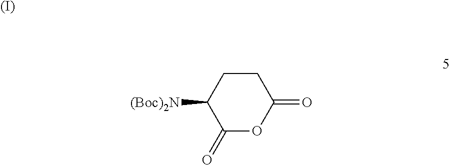

### CROSS REFERENCE TO RELATED APPLICATIONS

This application claims priority to U.S. Provisional Application No. 63/326,959, filed Apr. 4, 2022, the disclosure of which is incorporated herein by reference in its entirety.

### BACKGROUND

Controlling intracellular redox chemistry is an important aspect of homeostasis. Glutathione (GSH) is one of the key biochemicals controlling the thioredox state within cells. Glutathione is a tripeptide containing glutamic acid, cysteine and glycine in that order. Unlike most natural polypeptides, glutathione has the glutamic acid and cysteine bonded via the gamma-carboxylic acid of the glutamic acid. In addition to redox activity, glutathione is implicated in cell cycle regulation, proliferation, apoptosis, xenobiotic metabolism and thiol disulphide exchange. Glutathione is synthesized by two ATP dependent enzymes, glutamate cysteine ligase forms the γ-Glu-Cys amide yielding γ-glutamylcysteine and glutathione synthetase adds glycine.

Changes in GSH levels and/or redox state and chronic diseases is observed in multiple maladies such as neurogenerative diseases, diabetes, cystic fibrosis, HIV/AIDS and aging. GSH deficiencies manifest largely through increased vulnerability to oxidative stress. These observations have led some to conjecture that molecules which elevate glutathione levels would treat chronic and age-related disorders. Disease associated glutathione depletion appears to be controlled by the first biosynthetic enzyme glutamate cysteine ligase (GCL). Supporting this view, many diseases are related to impaired GCL activities. Ergo, supplementing γ-glutamylcysteine should increase cellular GSH levels. Because cytosolic concentrations of γ-glutamylcysteine are very low compared to Glutathione (7 μM) facile diffusion from extra- to intra-cellar environments is expected. Early studies demonstrated that γ-glutamylcysteine restores GSH concentrations in rodents. Oxidative injury in neurons and astrocytes and many other cells was reduced when γ-glutamylcysteine was delivered in vivo. [Redox Biology 2017, 11, 631-636] Overall, this analysis suggests that γ-glutamylcysteine could become a critical medicine.

Currently, γ-glutamylcysteine is produced in small quantities using both biochemical and chemical processes at per kilogram prices ranges from $1M-0.75M. To date, known methods of chemical synthesis provide low yields and involve two protection steps each for both cysteine and glutamic acid, one coupling step, and then at least two de-protection steps.

Another difficulty in the chemical synthesis of GGC is that selective protection of the α-carboxyl group of glutamic acid by esterification is complicated due to the higher reactivity of the γ-carboxyl group. The is also the possibility of racemization of any of the two chiral centers of GGC, which is a common problem in peptide synthesis.

The high intracellular concentrations of glutathione suggest that future therapies will require high dosing of γ-glutamylcysteine. Moving forward from here will require a synthesis process that can produce γ-glutamylcysteine for much lower prices.

Conventional processes for the synthesis of gamma L-glutamyl L-cysteine, bis gamma L-glutamyl cystine, and/or related target compounds are either enzymatic or biochemical methods which makes the process expensive and difficult for scaling up. Chinese patent no. CN1810769A (Synthesis process of gamma-L-glutamine-cysteine) discloses one strategy for the synthesis using phthalic anhydride as a protecting group. However, the removal of phthalic anhydride from the final product requires the use of hydrazine which is toxic and explosive.

Therefore, there is a need for an efficient high yield synthetic method for the preparation of γ-glutamylcysteine which can be scaled to industrial production quantities.

An object of this disclosure is to provide a cost-effective chemical synthesis method to prepare γ-glutamylcysteine in high yield and purity which is suitable for industrial-scale production.

### SUMMARY OF THE DISCLOSURE

This and other objects are obtained within the present disclosure, the first embodiment of which provides a method to prepare γ-L-Glutamyl-L-Cysteine or bis-γ-L-Glutamyl-L-Cystine, comprising:
preparing an aqueous solution of cysteine or cystine;
adding di-tert-butyl (S)-(2,6-dioxotetrahydro-2H-pyran-3-yl)iminodicarbonate (formula 5) to the aqueous solution of cysteine or cystine to obtain a reaction mixture;

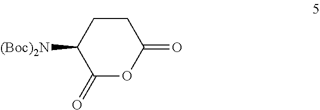

maintaining a pH of the reaction mixture of from 7 to 8 to obtain a γ ring-opening condensation of the cyteine or cystine amino group with the di-tert-butyl (S)-(2,6-dioxotetrahydro-2H-pyran-3-yl)iminodicarbonate;
isolating a bis(tert-butoxycarbonyl) protected γ-L-Glutamyl-L-Cysteine or bis-(tert-butoxycarbonyl) protected γ-L-Glutamyl-L-Cystine; and
hydrolyzing the bis(tert-butoxycarbonyl) groups to obtain γ-L-Glutamyl-L-Cysteine (formula 8) or bis-γ-L-Glutamyl-L-Cystine (formula 11)

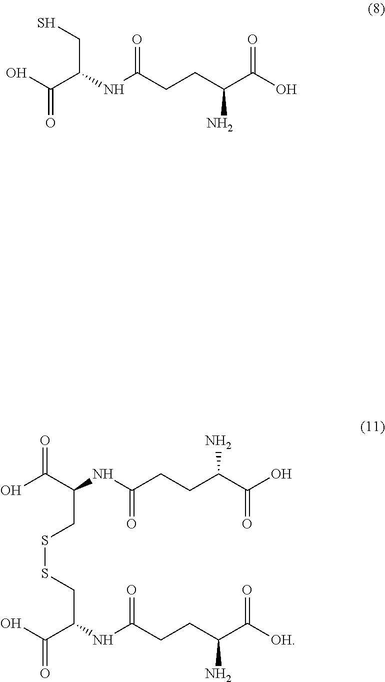 [SMILES: N[C@@H](CCC(=O)N[C@@H](CS)C(=O)O)C(=O)O ; N[C@@H](CCC(=O)N[C@@H](CSSC[C@H](NC(=O)CC[C@H](N)C(=O)O)C(=O)O)C(=O)O)C(=O)O]

In one aspect of the first embodiment the hydrolysis of the bis(tert-butoxycarbonyl) groups comprises reaction of the bis(tert-butoxycarbonyl) protected γ-L-Glutamyl-L-Cysteine or bis-(tert-butoxycarbonyl) protected γ-L-Glutamyl-L-Cystine with trifluoroacetic acid in an organic solvent. In a further aspect the γ-L-Glutamyl-L-Cysteine or bis-γ-L-Glutamyl-L-Cystine is isolated as a trifluoroacetate salt.

In another aspect of the first embodiment the method also comprises:
diesterification of L-glutamic acid;
isolation of the diester obtained;
reacting the amino group of the L-glutamic diester with 2 equivalents of di-tert-butyl dicarbonate [(Boc)₂O] to obtain an N,N-bis tert-butoxycarbonyl-L-glutamic diester;
hydrolyzing the diester groups to obtain N,N-bis(tert-butoxycarbonyl)-L-glutamic acid (formula 4);

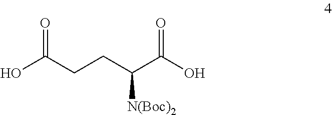

and
treating the N,N-bis(tert-butoxycarbonyl)-L-glutamic acid with acetic anhydride to obtain Di-tert-butyl (S)-(2,6-dioxotetrahydro-2H-pyran-3-yl)iminodicarbonate. In a further aspect the diesterification of L-glutamic acid comprises:
reacting both carboxylic acid groups L-glutamic acid with thionyl chloride in a primary alcohol solvent; and
removing the primary alcohol solvent to obtain the diester;
wherein the primary alcohol solvent is selected from the group consisting of methanol, ethanol, n-propanol and n-butanol.

The forgoing description is intended to provide a general introduction and summary of the present invention and is not intended to be limiting in its disclosure unless otherwise explicitly stated. The presently preferred embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a detailed synthesis scheme of γ-L-Glutamyl-L-Cysteine according to one embodiment of the present disclosure.

FIG. 2 shows a detailed synthesis scheme of bis-γ-L-Glutamyl-L-Cystine according to one embodiment of the present disclosure.

FIG. 3 shows possible nucleophilic ring opening positions for N-protected glutamic acidanhydride.

FIG. 4 shows the products obtained by reaction of L-cysteine with Cbz protected glutamic anhydride and Boc protected glutamic anhydride.

FIG. 5 shows the reaction scheme to obtain γ-L-Glutamyl-L-Cysteine by reaction of L-cysteine with DiBoc protected glutamic anhydride.

FIG. 6 shows the reaction scheme to obtain bis-γ-L-Glutamyl-L-Cystine by reaction of L-cystine with DiBoc protected glutamic anhydride.

### DETAILED DESCRIPTION

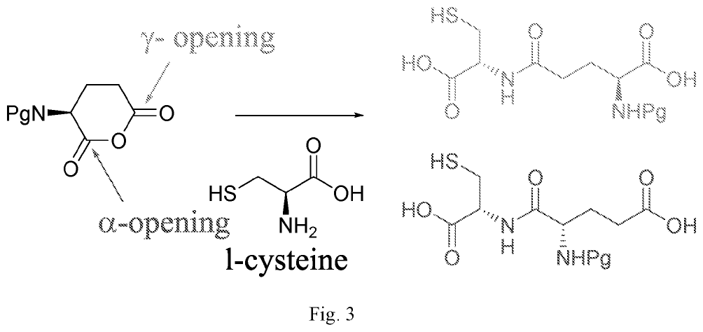

In addressing the problem of designing a chemical synthetic scheme for the preparation of γ-L-Glutamyl-L-Cysteine and bis-γ-L-Glutamyl-L-Cystine, the present inventors recognized that synthetically obtaining the desired target dipeptides in high yield and purity would require a regioselective nucleophilic ring opening of a N-protected anhydride of L-glutamic acid. The potential for α-ring opening and/or γ-ring opening is shown in FIG. 3 where “Pg” is a protecting group for the glutamyl amino N and L-cysteine is the nucleophilic specie.

The selectivity of the nucleophilic ring opening reaction of L-cysteine with a group of N-protected glutamic anhydrides was studied. Glutamic anhydrides having different N-protecting groups selected from t-butyloxycarbonyl (Boc), carboxybenzyl (Cbz) and bis t-butyloxycarbonyl (DiBoc) were prepared. The Cbz protected glutamic anhydride was synthesized in 95% yield from the corresponding Cbz protected glutamic acid. Similarly, the Boc protected anhydride was synthesized in 96% yield. DiBoc protected glutamic acid was prepared by first esterifying the carboxyl groups, treating the diester obtained stepwise with two equivalents of Di-tert-butyl dicarbonate to add two Boc groups to the amino N, hydrolyzing the diester and forming the anhydride ring from the free dicarboxylic acid groups. The DiBoc protected glutamic acid anhydride was obtained in 97% yield.

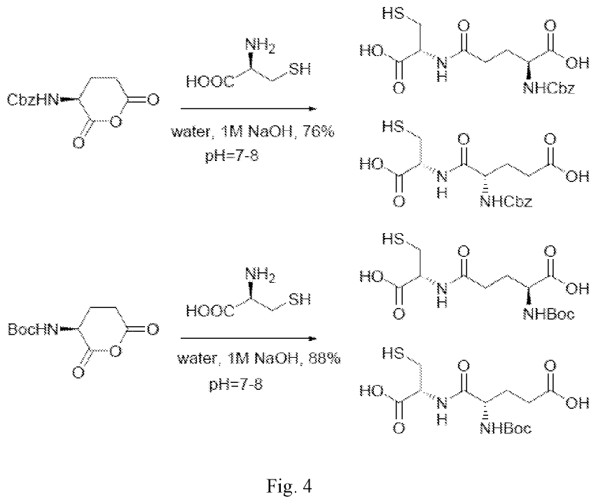

Treatment of the respective protected anhydrides with L-cysteine in water at pH 7-8 resulted in mixtures of α and γ ring opened products for the Cbz and Boc protected anhydrides as shown in FIG. 4. Reaction at a pH of 11-12 resulted in hydrolysis of the anhydride ring as well as the α and γ ring opened products.

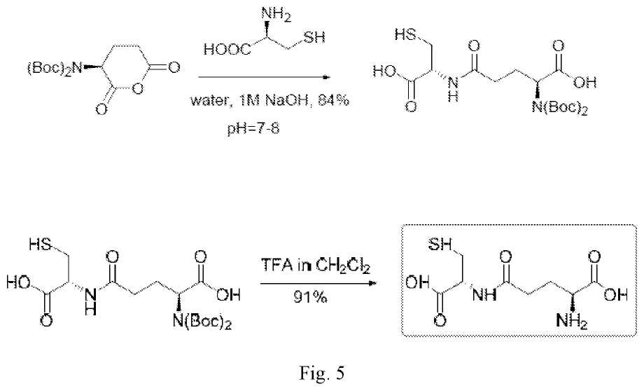

In contrast, treatment of the DiBoc protected glutamic acid anhydride with L-cysteine at pH 7-8 surprisingly yielded the γ ring opened product in 84%, and no α opening product was detected as shown in FIG. 5. Not wishing to be bound by theory, the inventors believe the high steric bulkiness of the DiBoc protection inhibits nucleophilic approach to the α position and thus, nucleophilic ring opening at the γ position is highly favored. Subsequent hydrolysis of the DiBoc groups with trifluoroacetic acid in methylene chloride yielded γ-L-Glutamyl-L-Cysteine in 91% yield.

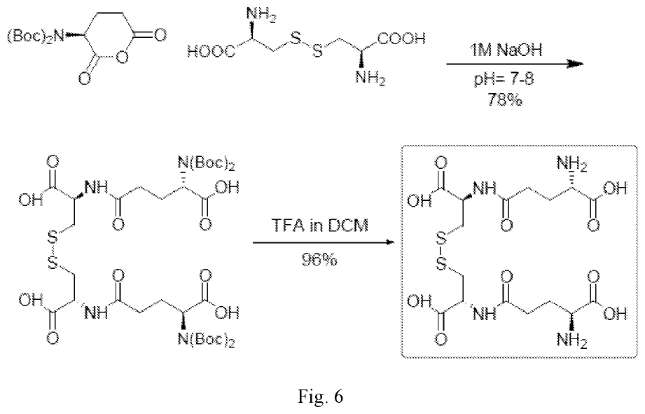

In view of the regioselectivity success obtained in the ring opening with L-cysteine as the nucleophile, the inventors investigated ring opening with L-cystine. As shown in FIG. 6 treatment of the DiBoc protected glutamic acid anhydride with L-cystine at pH 7-8 yielded the γ ring opened product in 78% yield, and no α opening product was detected. Hydrolysis of the DiBoc groups with trifluoroacetic acid in methylene chloride yielded bis-γ-L-Glutamyl-L-Cystine in 96%.

Thus, in a first embodiment, the present disclosure provides a method to prepare γ-L-Glutamyl-L-Cysteine or bis-γ-L-Glutamyl-L-Cystine, comprising:
preparing an aqueous solution of cysteine or cystine;
adding Di-tert-butyl (S)-(2,6-dioxotetrahydro-2H-pyran-3-yl)iminodicarbonate (formula 5) to the aqueous solution of cysteine or cystine to obtain a reaction mixture;

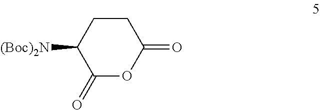

maintaining a pH of the reaction mixture of from 7 to 8 to obtain a γ ring-opening condensation of the cyteine or cystine amino group with the Di-tert-butyl (S)-(2,6-dioxotetrahydro-2H-pyran-3-yl)iminodicarbonate;
isolating a bis(tert-butoxycarbonyl) protected γ-L-Glutamyl-L-Cysteine or bis-(tert-butoxycarbonyl) protected γ-L-Glutamyl-L-Cystine; and
hydrolyzing the bis(tert-butoxycarbonyl) groups to obtain γ-L-Glutamyl-L-Cysteine (formula 8) or bis-γ-L-Glutamyl-L-Cystine (formula 11)

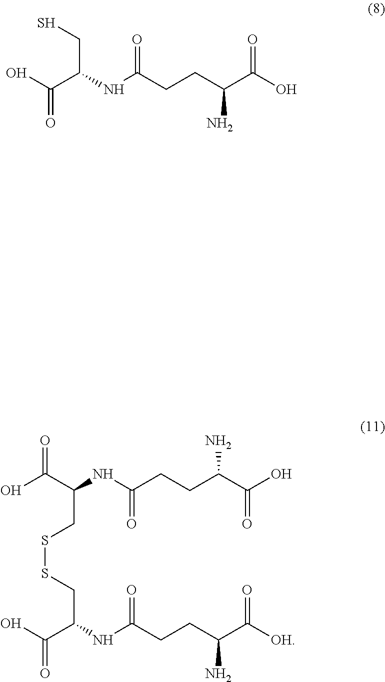 [SMILES: N[C@@H](CCC(=O)N[C@@H](CS)C(=O)O)C(=O)O ; N[C@@H](CCC(=O)N[C@@H](CSSC[C@H](NC(=O)CC[C@H](N)C(=O)O)C(=O)O)C(=O)O)C(=O)O]

The Di-tert-butyl (S)-(2,6-dioxotetrahydro-2H-pyran-3-yl)iminodicarbonate may be prepared by diesterification of L-glutamic acid with a primary alcohol, preferably a primary alcohol selected from the group consisting of methanol, ethanol, n-propanol and n-butanol. Most preferably, the primary alcohol is methanol. The method to prepare the diester is not limited and any conventionally known method to diesterify L-glutamic acid may be employed. In one method as described in the examples, the carboxylic acid groups may first be converted to acyl chloride groups with a chloroacylating agent such as POCl₂, PCl₅ or SOCl₂, and then reacted with the primary alcohol to obtain the diester. In a preferred aspect of the first embodiment, the L-glutamic acid may be dissolved in the anhydrous primary alcohol and then SOCl₂ may be added at a temperature of from −10° C. to 10° C., preferably −5° C. to 5° C. and most preferably, 0° C. Upon completion of the addition of the SOCl₂, the resulting reaction mixture may be heated to reflux temperature until the diester is obtained.

The progress of the diesterfication may be monitored by standard analytical methods including thin layer chromatography (TLC), high pressure liquid chromatrography (HPLC) and gas chromatography (GC). Such methods are conventionally known.

Upon completion of the diesterification the L-glutamate diester may be isolated by evaporation of the solvent or precipitation from the reaction mixture by addition of a solvent in which the diester is not soluble. The precipitate or residue obtained may be washed with additional non-solubilizing solvent or recrystallized.

The molar ratio of the chloroacylating agent to the L-glutamic acid may be from 2/1 to 5/1, preferably 2.1/1 to 4/1 and most preferably 2.5/1 to 3/1.

After isolation, the residue or precipitate of the L-glutamate diester obtained may be taken up in an anhydrous solvent, preferably an ether, most preferably tetrahydrofuran (THF), and treated with Di-tert-butyl dicarbonate ((Boc)₂O) in the presence of a tertiary amine, preferably, diisopropylethylamine. The initial addition of the (Boc)₂O to form the mono Boc protected L-glutamate diester may be conducted at a temperature of from −10° C. to 10° C., preferably −5° C. to 5° C. and most preferably, 0° C. The reaction mixture is then allowed to warm to 15° C. to 30° C. preferably, 18° C. to 27° C. and most preferably 25° C. and allowed to proceed to reaction completion. The progress of the reaction may be monitored by standard analytical methods including thin layer chromatography (TLC), high pressure liquid chromatrography (HPLC) and gas chromatography (GC). Such methods are conventionally known.

The mole ratio of the ((Boc)₂O) to the L-glutamate diester in the first stage may be from 1/1 to 3/1, preferably 1.05/1 to 2/1 and most preferably 1.2/1 to 1.5/1.

Upon completion of the first stage of N protection, the mono Boc protected L-glutamate diester may be isolated by evaporation of the solvent or precipitation from the reaction mixture by addition of a solvent in which the mono Boc protected product is not soluble. The precipitate or residue obtained may be washed with additional non-solubilizing solvent, recrystallized or dissolved in a solvent, preferably, ether. The solvent solution may be extracted with aqueous acid and/or aqueous base and dried. The solvent may then be evaporated to obtain the mono Boc protected L-glutamate diester.

In a second stage to form the di-Boc protected L-glutamate diester, the mono Boc protected L-glutamate diester may be dissolved in a fresh solvent, preferably acetonitrile (ACN) and treated with (Boc)₂O in the presence of a tertiary amine, preferably, dimethylaminopyridine (DMAP) at room temperature until the di-Boc protected L-glutamate diester is obtained. The progress of the reaction may be monitored by standard analytical methods including TLC, HPLC and GC.

Any solvent in which the mono Boc protected L-glutamate diester is soluble and which is non-reactive to (Boc)₂O and the tertiary amine may be employed for the second stage to form the di-Boc protected L-glutamate diester.

In the second stage of Boc addition the mole ratio of the ((Boc)₂O) to the mono Boc protected L-glutamate diester may be from 1.1/1 to 3/1, preferably 1.2/1 to 2/1 and most preferably 1.25/1 to 1.5/1.

The mole ratio of the tertiary amine to the mono Boc protected L-glutamate diester may be from 1/2 to 3/1, preferably 3/4 to 2/1 and most preferably 4/5 to 1/1.

The progress of the di-Boc protection reaction may be monitored by standard analytical methods including high pressure liquid chromatrography (HPLC) and gas chromatography (GC). Such methods are conventionally known.

Upon completion of the diBoc protection reaction the diBoc protected L-glutamate diester may be isolated by evaporation of the solvent or precipitation from the reaction mixture by addition of a solvent in which the di-Boc protected product is not soluble. The precipitate or residue obtained may be washed with additional non-solubilizing solvent or recrystallized.

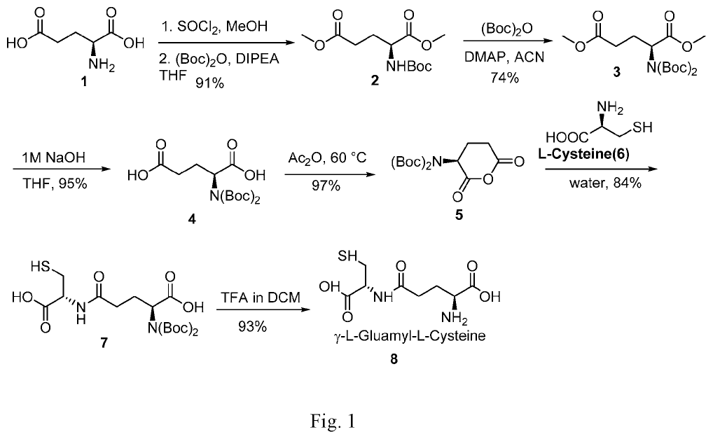

To obtain the Di-tert-butyl (S)-(2,6-dioxotetrahydro-2H-pyran-3-yl)iminodicarbonate (5) the diester groups of the diBoc protected L-glutamate diester are hydrolyzed to free carboxylic acids and then converted to the cyclic anhydride as shown in FIG. 1.

The diester hydrolysis may be conducted by dissolution of the N,N-bis(tert-butoxycarbonyl)-L-glutamate diester in an ether solvent, preferably THF, and treated with an aqueous solution of a hydroxide base, preferably NaOH. The progress of the hydrolysis may be monitored by standard analytical methods including TLC, HPLC and GC.

Upon completion of the hydrolysis the solvent may be removed by distillation or under vacuum and the resulting aqueous layer acidified and extracted multiple times with a solvent, preferably, ethyl acetate. The combined solvent extracts may be dried, for example over sodium sulfate, and the solvent removed under vacuum or by evaporation to obtain N,N-bis(tert-butoxycarbonyl)-L-glutamic acid (4)(FIG. 1).

The N,N-bis(tert-butoxycarbonyl)-L-glutamic acid may be converted to the cyclic anhydride of formula (5) by treatment with acetic anhydride under an inert atmosphere, preferably nitrogen at a temperature of from 30° C. to 80° C., preferably 40° C. to 75° C. and most preferably 50° C. to 70° C. The time required for formation of the cyclic anhydride depends upon the temperature and concentration of the starting N,N-bis(tert-butoxycarbonyl)-L-glutamic acid and may range fro 30 minutes to 6 hours. The progress of the reaction may be monitored by standard analytical methods including TLC. HPLC and GC.

Upon completion of the reaction to form the cyclic anhydride of formula (5) the reaction mixture may be cooled to a temperature of from 15° C. to 25° C. and the product Di-tert-butyl (S)-(2,6-dioxotetrahydro-2H-pyran-3-yl)iminodicarbonate (5) isolated by concentration and/or addition of a solvent in which the product is not soluble. The precipitate or residue obtained may be washed with additional non-solubilizing solvent, recrystallized, and filtered.

As described above, Di-tert-butyl (S)-(2,6-dioxotetrahydro-2H-pyran-3-yl)iminodicarbonate (5) is an important aspect of this disclosure due to the exclusive γ ring opening obtained when reacted with L-cycteine or L-cystine. This demonstrated regioselective reaction provides for the synthesis of γ-L-Glutamyl-L-Cysteine or bis-γ-L-Glutamyl-L-Cystine in high purity and high yield.

To prepare γ-L-Glutamyl-L-Cysteine, L-cysteine may be dissolved in water with an aqueous hydroxide base, preferably NaOH, and then the Di-tert-butyl (S)-(2,6-dioxotetrahydro-2H-pyran-3-yl)iminodicarbonate added to the solution. Reaction may be continued by addition of base to maintain the pH in the range 7-8. The progress of the reaction may be monitored by TLC, HPLC or GC. When the reaction is complete, N²,N²-Bis(tert-butoxycarbonyl)-N⁵—((R)-1-carboxy-2-mercaptoethyl)-L-glutamine (7) may be isolated by precipitation and filtration or by extraction with an organic solvent. In a preferred embodiment the aqueous reaction mixture is acidified to pH 3 and extracted with ethyl acetate (ETOAc). After drying, the EtOAc may be evaporated or concentrated to obtain the product.

The mole ratio of L-cysteine to the Di-tert-butyl (S)-(2,6-dioxotetrahydro-2H-pyran-3-yl)iminodicarbonate may be from 1/1 to 1/1.1 and preferably is 1/1 within a range of experimental error (±5%).

Although water is described as the solvent above, any solvent in which the L-cysteine and product (7) are readily soluble and from which the product is easily isolated may also be employed.

The isolated N²,N²—Bis(tert-butoxycarbonyl)-N⁵—((R)-1-carboxy-2-mercaptoethyl)-L-glutamine (7) may then be dissolved in an organic solvent, preferably CH₂Cl₂, and the di-Boc groups removed by reaction with trifluoroacetic acid (TFA). The TFA may be added at a temperature of 0° C. or lower and the reaction mixture allowed to warm to room temperature (20-25° C.). The progress of the reaction may be monitored by TLC, HPLC or GC. When the reaction is complete, the γ-L-Glutamyl-L-Cysteine may be isolated by removal of the solvent. The residue may be taken-up in a solvent and crystallized or the residue may be triturated with another non-solubilizing solvent to solidify or crystallize the product residue. The γ-L-Glutamyl-L-Cysteine may be isolated as a TFA salt.

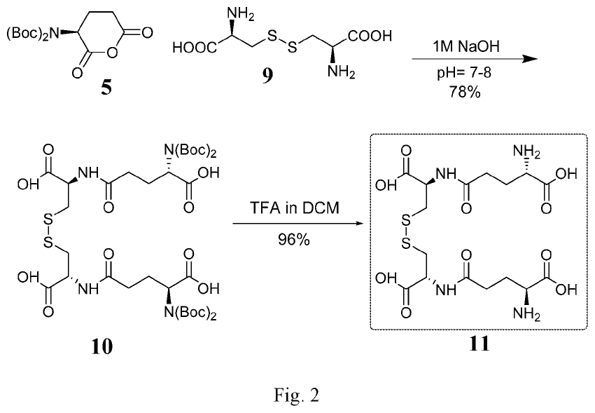

To prepare bis-γ-L-glutamyl-L-cystine, L-cystine may be dissolved in water with an aqueous hydroxide base, preferably NaOH, and then the Di-tert-butyl (S)-(2,6-dioxotetrahydro-2H-pyran-3-yl)iminodicarbonate added to the solution. Reaction may be continued by addition of base to maintain the pH in the range 7-8. The progress of the reaction may be monitored by TLC, HPLC or GC. When the reaction is complete, (6S,11R,16R,21S)-5,22-bis(tert-butoxycarbonyl)-2,2,25,25-tetramethyl-4,9,18,23-tetraoxo-3,24-dioxa-13,14-dithia-5,10,17,22-tetraazahexacosane-6,11,16,21-tetracarboxylic acid (10) (FIG. 2) may be isolated by precipitation and filtration or by extraction with an organic solvent. In a preferred embodiment the aqueous reaction mixture is acidified to pH 3 and extracted with ethyl acetate (ETOAc). After drying, the EtOAc may be evaporated or concentrated to obtain the product.

The mole ratio of L-cystine to the Di-tert-butyl (S)-(2,6-dioxotetrahydro-2H-pyran-3-yl)iminodicarbonate may be from 1/2 to 1/2.2 and preferably is 1/2 within a range of experimental error (±5%).

Although water is described as the solvent above, any solvent in which the L-cystine and product (10) are readily soluble and from which the product is easily isolated may also be employed.

The isolated (6S,11R,16R,21S)-5,22-bis(tert-butoxycarbonyl)-2,2,25,25-tetramethyl-4,9,18,23-tetraoxo-3,24-dioxa-13,14-dithia-5,10,17,22-tetraazahexacosane-6,11,16,21-tetracarboxylic acid (10) may then be dissolved in an organic solvent, preferably CH₂Cl₂, and the di-Boc groups removed by reaction with trifluoroacetic acid (TFA). The TFA may be added at a temperature of 0° C. or lower and the reaction mixture allowed to warm to room temperature (20-25° C.). The progress of the reaction may be monitored by TLC, HPLC or GC. When the reaction is complete, the bis-γ-L-Glutamyl-L-Cystine may be isolated by removal of the solvent. The residue may be taken-up in a solvent and crystallized or the residue may be triturated with another non-solubilizing solvent to solidify or crystallize the product residue. The bis-γ-L-Glutamyl-L-Cystine may be isolated as a TFA salt.

### EXAMPLES

### I. Synthesis OF γ-L-Glutamyl-L-Cysteine

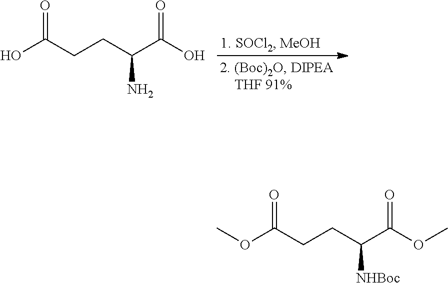

Dimethyl (tert-butoxycarbonyl)-L-glutamate (2): To a solution of L-glutamic acid (15 g, 102 mmol) in anhydrous MeOH (120 mL) was added SOCl₂ (29.6 mL, 407 mmol) drop-wise at 0° C. the reaction mixture was heated to reflux for 2 h and cooled to room temperature, and then the solvent was evaporated. The residue obtained was taken in anhydrous THF (120 mL), (Boc)₂O (26.7 g, 122 mmol) and diisopropyl ethylamine (35.5 mL, 204 mmol) were added dropwise at 0° C. Then the reaction mixture was allowed to warm to RT and stirred for 5 h. Solvent was evaporated and the obtained solid was dissolved in ether (120 mL). The organic layer was washed with 2N HCl (60 mL×2), H₂O (60 mL×2), saturated NaHCO₃ solution (60 mL×2) and brine (60 mL×2). The organic phase was dried over Na₂SO₄, concentrated to afford product 2 (25.6 g, 91%) as a colorless oil, which was become solid after 18-20 h on standing. ¹H NMR (600 MHz, CDCl3) δ: 4.22 (d, J=4.7 Hz, 1H), 3.64 (s, 3H), 3.57 (s, 3H), 2.41-2.22 (m, 2H), 2.08 (td, J=13.1, 7.0 Hz, 1H), 1.86 (td, J=14.7, 8.1 Hz, 1H), 1.33 (s, 9H); 13C NMR (150 MHz, CDCl3) δ. 173.00, 172.55, 155.30, 79.68, 52.77, 52.18, 51.56, 29.94, 28.05, 27.49.

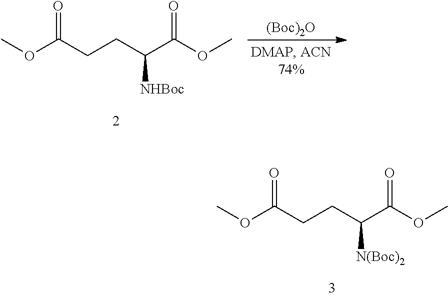

Dimethyl N,N-bis(tert-butoxycarbonyl)-L-glutamate (3): To a solution of compound 2 (25 g, 90.8 mmol) in ACN (150 mL). DMAP (8.88 g, 72.6 mmol) and Boc2O (23.8 g, 109 mmol) were added and stirred at room temperature for 5 h. An additional amount of Boc₂O (8 g, 36 mmol) and DMAP (2.2 g, 18) mmol were added and the reaction was stirred at room temperature for 16-18 h. The solvent was removed in vacuo and the crude product allowed to stand for 24 h. Solid formation was observed, pentane (100 mL) was added and filtered using a Buchner Funnel and dried to afford compound 3 (25.3 g, 74%) as white crystalline solid.

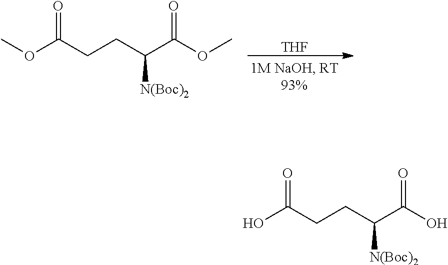

N,N-bis(tert-butoxycarbonyl)-L-glutamic acid (4): To a stirred solution of dimethyl N,N-bis(tert-butoxycarbonyl)-L-glutamate (34 g, 90.6 mmol) in THF (200 mL), 1M NaOH (190 mL, 190 mmol) was added drop wise for 10-15 min at 0° C. and stirred for 40 h. The reaction was monitored by TLC (50% ethyl acetate in hexanes) showed complete consumption of starting material. THF was removed under vacuum and aqueous layer was washed with Et2O (50 mL). Separated the aqueous layer, acidified to pH 3 using 2N HCl and extracted with EtOAc (100 mL×2). Combined organic fractions were dried over sodium sulfate and concentrated under vacuo to afford N, N-bis(tert-butoxycarbonyl)-L-glutamic acid (4) as off-white solid (30.2 g, 96%). ¹H NMR (600 MHz, DMSO-d₆): δ 12.44 (br. s., 2H), 4.81-4.66 (m, 1H), 2.33-2.15 (m, 3H), 2.06-1.89 (m, 1H), 1.42 (s, 18H); ¹³C NMR (150 MHz, DMSO-d₆): δ 174.1, 172.0, 152.1, 82.8, 57.6, 30.6, 28.0, 25.2.

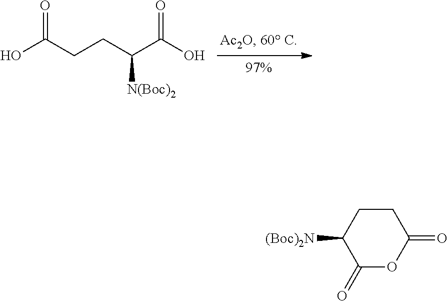

Di-tert-butyl (S)-(2,6-dioxotetrahydro-2H-pyran-3-yl)iminodicarbonate (5): A mixture of N,N-bis(tert-butoxycarbonyl)-L-glutamic acid 4 (30 g, 86 mmol) and acetic anhydride (150 mL) was heated at 60° C. under nitrogen for 2 h. The reaction mixture was cooled to room temperature toluene (90 mL) was added then concentrated in vacuo. White solid was obtained, pentane (100 mL) was added and filtered using a Büchner Funnel and dried. Compound 5 was obtained as a white solid (27.5 g, 97%). ¹H NMR (600 MHz, DMSO-d₆): δ 5.35 (dd, J=6.1, 12.2 Hz, 1H), 3.03 (ddd, J=5.9, 14.3, 17.1 Hz, 1H), 2.86-2.79 (m, 1H), 2.33-2.24 (m, 1H), 2.06-1.99 (m, 1H), 1.45 (s, 18H); ¹³C NMR (150 MHz, DMSO-d₆): δ 167.0, 166.8, 151.6, 84.0, 54.7, 29.6, 28.0, 21.7.

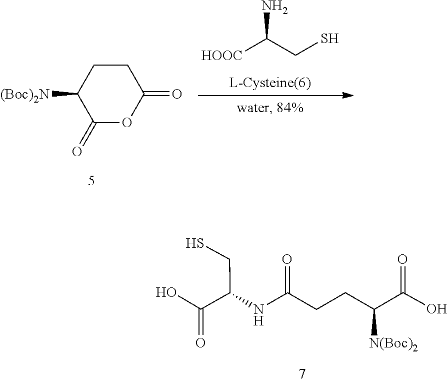

N²,N²-Bis(tert-butoxycarbonyl)-N⁵—((R)-1-carboxy-2-mercaptoethyl)-L-glutamine (7): L-Cysteine (3.7 g, 30.4 mmol) was taken in water (20 mL). 1M NaOH (30.4 mL, 30.4 mmol) was added and stirred till the solids were dissolved. The compound 5 (10 g, 30.4 mmol) was added and continued the stirring. Another portion of 1M NaOH (30.4 mL, 30.4 mmol) was added over 2-3 h to the reaction mixture by maintaining the pH 7-8. The reaction mixture becomes clear solution. Washed the reaction mixture with EtOAc (30 mL), the aqueous layer was acidified to pH 3 using 2N HCl and extracted with EtOAc (50 mL×2). Combined organic layers were dried over sodium sulfate concentrated under vacuum to afford desired compound 7 (11.5 g, 84%) as off-white solid.

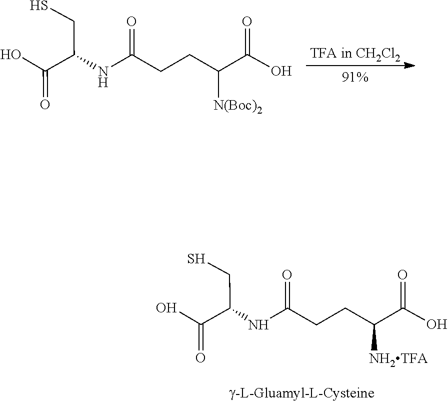

(S)-5-(((R)-1-carboxy-2-mercaptoethyl)amino)-5-oxo-2-((2,2,2-trifluoroacetyl)-14-azaneyl)pentanoic acid (gamma L-glutamyl L-cysteine) 8: To a solution of compound 7 (17 g, 37.7 mmol) in CH₂Cl₂ (100 mL), trifluoroacetic acid (17.3 mL, 226.4 mmol) was added at 0° C. and allowed the reaction mixture to warm to room temperature. Reaction mixture was stirred for 3 h at room temperature and CH₂Cl₂ was removed under vacuum. Resulted reaction mixture was titruated with diethyl ether (50 mL) and dried again under vacuum until nice solid forms. The desired product gamma L-glutamyl L-cysteine was obtained as a TFA salt (12.2 g, 93%).

### II. Synthesis of Bis-γ-L-Glutamyl-L-Cystine

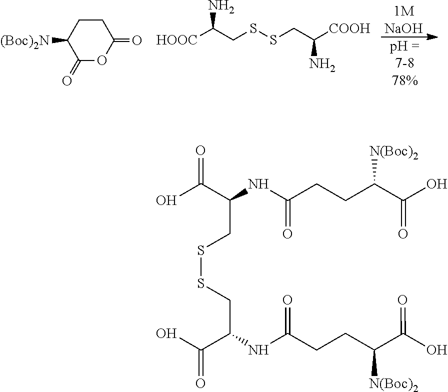

L-Cystine, 9 (2.55 g, 10.6 mmol) was take in water (5 mL). 1M NaOH (21.2 mL) was added and stirred till the solids were dissolved. The compound 5 (7.0 g, 21.3 mmol) was added and continued the stirring. Another portion of 1M NaOH (21.2 mL) was added over 2-3 h to the reaction mixture by maintaining the pH 7-8. The reaction mixture becomes clear solution. Washed the reaction mixture with EtOAc (25 mL), the aqueous layer was acidified to pH 3 using 2N HCl and extracted with EtOAc (50 mL×2). Combined organic layers were dried over sodium sulfate concentrated under vacuum to afford desired compound 10 as off-white solid (14.9 g, 78%).

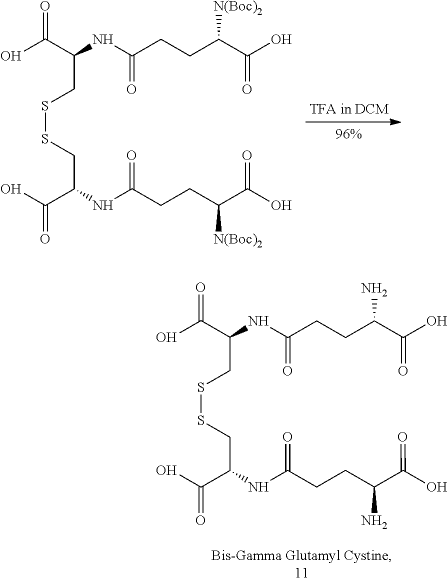

(4S,9R,14R,19S)-1,1,1,22,22,22-hexafluoro-2,7,16,21-tetraoxo-11,12-dithia-314,8,15,2014-tetraazadocosane-4,9,14,19-tetracarboxylic acid (bis gamma L-glutamyl L-cystine) 11: To a solution of compound 10 (9 g, 10 mmol) in CH₂Cl₂ (80 mL), trifluoroacetic acid (16 mL) was added at 0° C. and allowed the reaction mixture to warm to room temperature. Reaction mixture was stirred for 3 h at room temperature and CH₂Cl₂ was removed under vacuum. Resulting reaction mixture was titruated with diethyl ether (50 mL) and dried again under vacuum until nice solid forms. The desired product bis gamma L-glutamyl L-cystine 11 was obtained as a TFA salt (6.65 g, 96%).

The above description is presented to enable a person skilled in the art to make and use the embodiments and aspects of the disclosure and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein. In this regard, certain embodiments within the disclosure may not show every benefit of the disclosure, considered broadly.

1. A method to prepare γ-L-Glutamyl-L-Cysteine or bis-γ-L-Glutamyl-L-Cystine, comprising:
preparing an aqueous solution of cysteine or cystine;
adding Di-tert-butyl (S)-(2,6-dioxotetrahydro-2H-pyran-3-yl)iminodicarbonate (formula 5) to the aqueous solution of cysteine or cystine to obtain a reaction mixture; 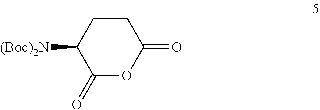
maintaining a pH of the reaction mixture of from 7 to 8 to obtain a γ ring-opening condensation of the cyteine or cystine amino group with the Di-tert-butyl (S)-(2,6-dioxotetrahydro-2H-pyran-3-yl)iminodicarbonate;
isolating a bis(tert-butoxycarbonyl) protected γ-L-Glutamyl-L-Cysteine or bis-(tert-butoxycarbonyl) protected γ-L-Glutamyl-L-Cystine; and
hydrolyzing the bis(tert-butoxycarbonyl) groups to obtain γ-L-Glutamyl-L-Cysteine (formula 8) or bis-γ-L-Glutamyl-L-Cystine (formula 11)

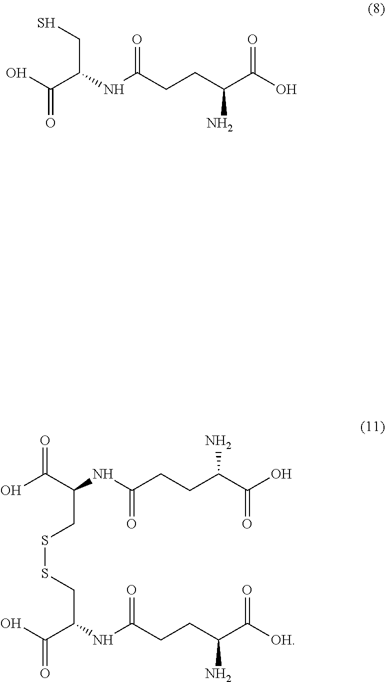 [SMILES: N[C@@H](CCC(=O)N[C@@H](CS)C(=O)O)C(=O)O ; N[C@@H](CCC(=O)N[C@@H](CSSC[C@H](NC(=O)CC[C@H](N)C(=O)O)C(=O)O)C(=O)O)C(=O)O]

2. The method of claim 1, wherein hydrolysis of the bis(tert-butoxycarbonyl) groups comprises reaction of the bis(tert-butoxycarbonyl) protected γ-L-Glutamyl-L-Cysteine or bis-(tert-butoxycarbonyl) protected γ-L-Glutamyl-L-Cystine with trifluoroacetic acid in an organic solvent.

3. The method of claim 2, wherein the γ-L-Glutamyl-L-Cysteine (formula 8) or bis-γ-L-Glutamyl-L-Cystine (formula 11) is isolated as a trifluoroacetate salt.

4. The method of claim 1, further comprising:
diesterification of L-glutamic acid;
isolation of the diester obtained;
reacting the amino group of the L-glutamic diester with 2 equivalents of di-tert-butyl dicarbonate [(Boc)₂O] to obtain an N,N-bis tert-butoxycarbonyl-L-glutamic diester;
hydrolyzing the diester groups to obtain N,N-bis(tert-butoxycarbonyl)-L-glutamic acid (formula 4); 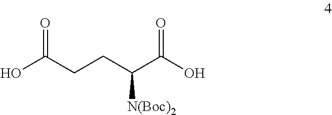
and
treating the N,N-bis(tert-butoxycarbonyl)-L-glutamic acid with acetic anhydride to obtain Di-tert-butyl (S)-(2,6-dioxotetrahydro-2H-pyran-3-yl)iminodicarbonate.

5. The method of claim 4, wherein the diesterification of L-glutamic acid comprises:
reacting both carboxylic acid groups L-glutamic acid with thionyl chloride in a primary alcohol solvent; and
removing the primary alcohol solvent to obtain the diester;
wherein the primary alcohol solvent is selected from the group consisting of methanol, ethanol, n-propanol and n-butanol.
